SEQUENCE LISTING (1 sequence)

SEQ ID NO: 1 - protein, 450 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0353876 A1
(43) Pub. Date: Nov. 20, 2025

(54) GENTLE AND DIRECT COPPER-BASED PROTEIN AZIDYLATION FOR BIOCONJUGATION

(71) Applicant: Wisconsin Alumni Research Foundation, Madison, WI (US)

(72) Inventors: Michael Sussman, Cross Plains, WI (US); Benjamin Minkoff, Madison, WI (US); Jamison Wolfer, Cross Plains, WI (US); Yun Hu, Madison, WI (US); Jennifer Schomaker, New Glarus, WI (US)

(73) Assignee: Wisconsin Alumni Research Foundation, Madison, WI (US)

(21) Appl. No.: 19/206,755

(22) Filed: May 13, 2025

Related U.S. Application Data
(60) Provisional application No. 63/649,605, filed on May 20, 2024.

(51) Int. Cl. C07K 1/107

(52) U.S. Cl. CPC C07K 1/1077

(57) ABSTRACT

A method of attaching an azide moiety to a biomolecule. The method comprises contacting a biomolecule in a solution with an azide and a copper, for a time wherein at least one azide moiety is covalently bonded to the biomolecule to yield an azidylated biomolecule. The copper is copper (I), and can be generated from copper (II) by a reductant. The solution further comprises a copper ligand for reducing degradation of the biomolecule. The azilylated biomolecule can be attached to a reagent comprising an alkyne via a copper-catalyzed azide-alkyne cycloaddition (“CuAAC”) reaction or a strain-promoted alkyne-azide cycloaddition (“SPAAC”) reaction.

### CROSS-REFERENCE TO RELATED APPLICATIONS

Priority is hereby claimed to provisional application Ser. No. 63/649,605, filed May 20, 2024, which is incorporated herein by reference.

### FEDERAL FUNDING STATEMENT

This invention was made with government support under DE-FG02-88ER13938 awarded by the US Department of Energy and under 2010789 and 2203611 awarded by the National Science Foundation. The government has certain rights in the invention.

### REFERENCE TO AN ELECTRONIC SEQUENCE LISTING

The contents of the electronic sequence listing (sequencelistP240219US02.xml; Size: 2.34 kilobytes; and Date of Creation: May 9, 2025) is herein incorporated by reference in its entirety.

### BACKGROUND

The development of so-called “click chemistry,” e.g., copper-catalyzed azide-alkyne cycloaddition (“CuAAC”), in the early 2000′s opened a new era in the study of molecular interactions. See, for example, Baskin J M, Bertozzi C R. (2007) “Bioorthogonal Click Chemistry: Covalent Labeling in Living Systems,” QSAR Comb Sci. 26:1211-1219.

Click chemistry provides an easy way to covalently link molecules together, and has proven to be well-suited to biomolecular investigations. See, for example, Presolski, Hong, and Finn (2011) “Copper-Catalyzed Azide-Alkyne Click Chemistry for Bioconjugation,” Curr Protoc Chem Biol. 3(4):153-162. As noted there, azides and alkynes are small and unobtrusive moieties. They lack the ability to engage in strong hydrogen bonding, as well as acid-base, hydrophobic, coulombic, dipolar, and x-stacking interactions. As a result, they minimally perturb the biological molecules to which they are attached (if at all). The literature now includes a growing number of examples in which azide-or alkyne-derivatized nutrients or cofactors are taken up and incorporated into biological molecules by living cells. By way of a very small sampling, see Kiick K L, Saxon E, Tirrell D A, Bertozzi C R (2002) “Incorporation of Azides into Recombinant Proteins for Chemoselective Modification by the Staudinger Ligation,” Proc Natl Acad Sci U S A. 99(1):19-24; Ning X, Guo J, Wolfert M A, Boons G J (2008) “Visualizing Metabolically Labeled Glycoconjugates of Living Cells by Copper-Free and Fast Huisgen Cycloadditions,” Angew Chem Int Ed Engl. 47(12):2253-5; and Rangan K J, Yang Y Y, Charron G, Hang H C (2010) “Rapid visualization and large-scale profiling of bacterial lipoproteins with chemical reporters,” J Am Chem Soc. 132:10628-1062.

An early description of the CuAAC reaction is found in Kolb H C, Finn M G, Sharpless K B (2001) “Click Chemistry: Diverse Chemical Function from a Few Good Reactions,” Angewandte Chemie International Edition 40(11):2004-2021. Many discoveries, tools, and products have been developed from that initial discovery. Covalently attaching molecules together with click chemistry, however, requires first synthesizing or attaching the reactive azide and alkyne moieties to the molecules that are to be linked or “clicked.” This has proven problematic when working with macromolecular biomolecules such as whole proteins, macromolecular nucleic acids, and the like.

The uncatalyzed 1,3-dipolar cycloaddition reaction of standard azides and alkynes is highly specific, but quite slow without catalysis. The Cu(I) catalysis of the reaction between azides and terminal alkynes was first described independently in 2002 in Tornøe C W, Christensen C, Meldal M. (2002) “Peptidotriazoles on Solid Phase: [1,2,3]-Triazoles by Regiospecific Copper(I)-Catalyzed 1,3-Dipolar Cycloadditions of Terminal Alkynes to Azides,” J. Org. Chem. 67:3057-3062; and Rostovtsev V V, Green L G, Fokin V V, Sharpless K B (2002) “A Stepwise Huisgen Cycloaddition Process: Copper(I)-Catalyzed Regioselective Ligation of Azides and Terminal Alkynes,” Angew Chem, Int Ed. 41:2596-2599. Another general solution to the azide-alkyne reaction rate problem is to make the alkyne highly strained in a ring structure. Such reactions are now referred to as “copper-free click chemistry.” See, for example, Codelli J A, Baskin J M, Agard N J, Bertozzi C R (2008) “Second-Generation Difluorinated Cyclooctynes for Copper-Free Click Chemistry,” J. Am. Chem. Soc. 130 (34): 11486-11493.

As flexible and useful as it is, click chemistry still requires that one of the molecules contains an azide and the other an alkyne. As noted above, affixing one or the other of these reactive groups to biomolecules has proven troublesome. Thus, there remains a long-felt and unmet need for a fast, easy, and direct method to azidylate biomolecules, including proteins, polypeptides, and nucleic acids. Such a method would enable access to the panoply of reactions that can be accomplished using click chemistry.

### SUMMARY

Disclosed herein is an orthogonal reaction for direct protein azidylation. The reaction requires only copper(I), azide, and protein. Unlike with oxidative azidylation, which appeared to modify many residues, this system, due to the Cu(I) as a major reactant, may be directed to and modify Histidine residues, based upon literature precedent for His:Cu(I) coordination. Thus, this presents a more controllable system for protein modification. This method enables click chemistry directly on proteins, and thus may have value on top of the current azidylation methods for use in bioconjugation.

Specifically, disclosed herein is a method of attaching an azide moiety to a biomolecule, the method comprising contacting a biomolecule in a solution with an azide and a copper, for a time wherein at least one azide moiety is covalently bonded to the biomolecule to yield an azidylated biomolecule.

In one version, the copper is copper(I). Alternatively, the copper is copper(II), and the solution comprises a reductant to generate copper(I) from copper(II).

The solution may further comprise a ligand to coordinate the copper for reducing degradation of the biomolecule.

The biomolecule may be a protein, and by way of non-limiting, the protein may be selected from the group consisting of an intracellular protein, a membrane-bound protein, a circulating protein, and an antibody. Alternatively, the biomolecule may be a nucleic acid polymer, such as a DNA or RNA polymer.

The contact time of the biomolecule with the azide is not limiting. It can range from about 1 second to about 20 hours.

The method further comprises reacting the azidylated biomolecule with a reagent comprising an alkyne. The alkyne may be a terminal alkyne or an internal alkyne. The reagent comprising an alkyne may be a cyclic alkyne. The reaction with the alkyne may be a copper-catalyzed azide-alkyne cycloaddition (“CuAAC”) reaction or a strain-promoted alkyne-azide cycloaddition (“SPAAC”) reaction.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1 shows azidylation of Trastuzumab with or without Cu, TEMPO, and azide. The reaction was conducted with 1.24 μM Trastuzumab, +/−10 mM sodium azide (NaN₃), +/−500 μM TEMPO, +/−500 μM Copper(I) Chloride (Cl(I)Cl), and 0.5% acetonitrile (from Cu(I)Cl dilution). The reaction was run for 20 min and the products were desalted with Zeba spin desalting columns. Fluorescent alkyne was subsequently supplied to the reaction and clicked for 20 min with standard Cu click reaction. Gel results were shown. Top is Coomassie protein stain to assess protein load and/or degradation, and bottom is fluorescent image of the same gel, to assess covalent azide addition (azidylation).

FIG. 2 shows azidylation of Trastuzumab using Cu(I) or Cu(II) with or without ligands. The reaction was conducted with 1.24 μM Trastuzumab, +/−5 mM NaN₃, +500 μM Cu(I)Cl or Cu(II)Sulfate supplied as free Cu(I) or Cu(II) salt or bound to the ligand THTPA or BTTAA, and 0.5% acetonitrile. The products were desalted with Zeba spin desalting columns. Fluorescent alkyne was subsequently supplied to the reaction and clicked for 20 min with standard Cu click reaction.

FIG. 3 shows azidylation of Trastuzumab with 0.5, 1, or 2 mM Cu(I) and 15 or 30 min reaction time. The reaction was conducted with 1.24 μM Trastuzumab, +/−10 mM NaN₃, +0.5 mM, 1 mM, or 2 mM Cl(I)Cl, and 0.5%, 1%, or 2% Acetonitrile (from 100 mM Cu(I)Cl dilution). The products were desalted with Zeba spin desalting columns. Fluorescent alkyne was subsequently supplied to the reaction and clicked for 20 min with standard Cu click reaction.

FIG. 4 shows azidylation of Trastuzumab with DMSO as solvent. The reaction was conducted with 1.24 μM Trastuzumab, +/−5 mM NaN₃, +/−500 μM Cu(I)Cl, and 1.5%, 5.5%, or 10.5% DMSO. The products were desalted with Zeba spin desalting columns. Fluorescent alkyne was subsequently supplied to the reaction and clicked for 20 min with standard Cu click reaction.

FIG. 5 shows azidylation of Trastuzumab with four different forms of Cu(I), including tetrakis (acetonitrile) copper(I) hexafluorophosphate (C1), tetrakis (acetonitrile) copper(I) trifluoromethanesulfonate (C2), copper(I) iodide (C3), and copper(I) chloride (C4). The reaction was conducted with 1.24 μM Trastuzumab, +/−5 mM NaN₃, +/−500 μM C1, C2, C3, or C4, and 0.5% organic solvent (either DMSO or acetonitrile). The reaction was run for 20 min.

FIG. 6 shows azidylation of Trastuzumab in different buffers, including water, 25 or 50 mM ammonium bicarbonate (AMBIC), 50 mM Tris-HCl, and 50 mM sodium phosphate (NaPO₄). The reaction was conducted with 1.24 μM Trastuzumab, +/−5 mM NaN₃, +/−500 M Cu(I)Cl, and 0.5% acetonitrile with the different buffers.

FIG. 7 shows azidylation of Trastuzumab with TMSN₃ as an azide donor compared to NaN₃. Tris-HCl and triethyl ammonium bicarbonate (TEAB) were tested due to different number of alcohol groups to scavenge potential silylation of protein sidechains and gauge reaction efficiency. The reaction was conducted with 1.24 μM Trastuzumab, +/−5 mM NaN₃ or 1 mM/5 mM TMSN₃, +/−500 μM Cu(I)Cl, 0.5% acetonitrile, and Tris-HCl or TEAB as background buffer (˜pH8.5).

FIG. 8 shows an annotated amino acid sequence (SEQ ID NO:1) compiled from mass spectrometry results confirming direct azide addition to Trastuzumab (sold under the registered trademark “HERCEPTIN”®, Genentech, Inc. San Francisco, California) and subsequently attaching a derivatized drug alkyne via click chemistry. The azide modification occurred at the two bold residues at positions 271 and 272. The ultimate modification was a triazole group linked to Exatacan (i.e., (1S,9S)-1-amino-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione.)

FIG. 9 shows azidylation of Trastuzumab by premixing Cu(I)Cl and TMSN₃ before adding to protein versus adding Cu(I)Cl first for 1 min, then adding TMSN₃. This is to test whether the order of adding reagents could impact the reaction. The reaction was conducted with 1.24 μM Trastuzumab, +/−5 mM NaN₃ or 1 mM/5 mM TMSN₃, +/−100 μM or 500 μM Cu(I)Cl, and 10% acetonitrile, all in 50 mM TEAB.

FIG. 10 shows azidylation of Trastuzumab using CuI or CuCl and 10% or 20% acetonitrile. The reaction was conducted with 1.24 μM Trastuzumab, +/−5 mM NaN₃ or TMSN₃, +/−100 μM Cu(I)Cl or Cu(I)I, and 10% or 20% acetonitrile, all in 50 mM TEAB.

FIG. 11 shows azidylation of Trastuzumab with different concentrations of TMSN₃ and Cu(I). The reaction was conducted with 1.24 μM Trastuzumab, +1 mM or 5 mM TMSN₃, +12.5, 25, 50, or 100 μM CuI, and 10% acetonitrile, all in 50 mM TEAB.

FIG. 12 shows an exemplary reaction of protein azidylation by TMSN₃ and with ascorbate to regenerate Cu(I) from Cu(II).

FIG. 13 shows azidylation of Trastuzumab comparing conditions with Cu(I) (as copper iodide) vs. Cu(II) (as cupric sulfate), with or without sodium ascorbate to generate Cu(I) from Cu(II) in situ, and with or without the ligand THPTA. The reaction was conducted with 1.37 μM Trastuzumab, 50 mM TEAB (˜pH 8.5), +/−5 mM sodium ascorbate in water, +/−100 μM Cu(I) or Cu(II), +/−500 M THPTA, and 5 mM TMSN₃, all in 10% acetonitrile due to TMSN₃ solubility. The reaction was run for 30 min.

FIG. 14 shows azidylation of Trastuzumab with titrating down added Cu(II) and THPTA while keeping TMSN₃ and ascorbate constant, and running the reaction twice as long as the test shown in FIG. 13 (30 min vs. 60 min). Cu:THPTA ratio is kept constant at 1:5. The reaction was conducted with 1.37 μM Trastuzumab, 50 mM TEAB (˜pH 8.5), +1 or 5 mM sodium ascorbate in water, +10, 50, or 100 μM Cu(II) (supplied as cupric sulfate), +50, 250, or 500 μM THPTA, and 5 mM TMSN3, all in 10% acetonitrile due to TMSN₃ solubility.

FIG. 15 shows azidylation of Trastuzumab with lower concentrations of Cu(II) (1 μM) and THPTA (5 μM) based on the test results shown in FIG. 14, and the reaction was run overnight for about 17 hours. The Cu:THPTA ratio is kept constant at 1:5. The reaction was conducted with 1.37 μM Trastuzumab, 50 mM TEAB (˜pH 8.5), +1 or 5 mM sodium ascorbate in water, +1, 10, 50, or 100 μM Cu(II) (supplied as cupric sulfate), +5, 50, 250. or 500 μM THPTA, and 5 mM TMSN₃, all in 10% acetonitrile due to TMSN₃ solubility.

FIG. 16 shows relative intensities of bands as shown in lanes 1-5 of FIG. 15, comparing protein azidylation with 1, 10, 50, and 100 μM Cu(II) added.

FIG. 17 shows azidylazed Trastuzumab without click reaction, or clicking with standard CuAAC reagent, ½×, or ¼× CuAAC reagents. The azidylation was conducted with TMSN₃, Cu(II), and ascorbate overnight.

FIG. 18 shows CuAAC vs. SPAAC reactions of azidylazed Trastuzumab. The CuAAC reaction was run for 30 min, and the SPAAC reaction was run for 1, 5, or 45 hours. The azidylation of Trastuzumab prior to SPAAC reaction was conducted with 100 or 500 μM ascorbate and compared to no azide control.

FIG. 19 is a gel showing that His-tagged human serum albumin (HSA) is modified more heavily using the disclosed method than is untagged HSA. See examples for experimental details.

FIG. 20 is a gel showing that the results depicted in FIG. 19 are reproducible and quantifiable using dibenzocyclooctyne-poly (ethylene) glycol (DBCO-PEG) click reagents.

### DETAILED DESCRIPTION

### Abbreviations and Definitions

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations shall include the corresponding plural characteristic or limitation, and vice versa, unless otherwise specified or clearly implied to the contrary by the context in which the reference is made. That is, unless specifically stated to the contrary, “a” and “an” mean “one or more.” The phrase “one or more” is readily understood by one of skill in the art, particularly when read in context of its usage. For example, “one or more” substituents on a phenyl ring designates one to five substituents.

All combinations of method or process steps as used herein can be performed in any order, unless otherwise specified or clearly implied to the contrary by the context in which the referenced combination is made.

The methods disclosed herein can comprise, consist of, or consist essentially of the essential elements and limitations of the method as described herein, as well as any additional or optional ingredients, components, or limitations described herein or otherwise useful in synthetic organic chemistry. The disclosure provided herein suitably may be practiced in the absence of any element which is not specifically disclosed herein.

### Chemical Names and Structures

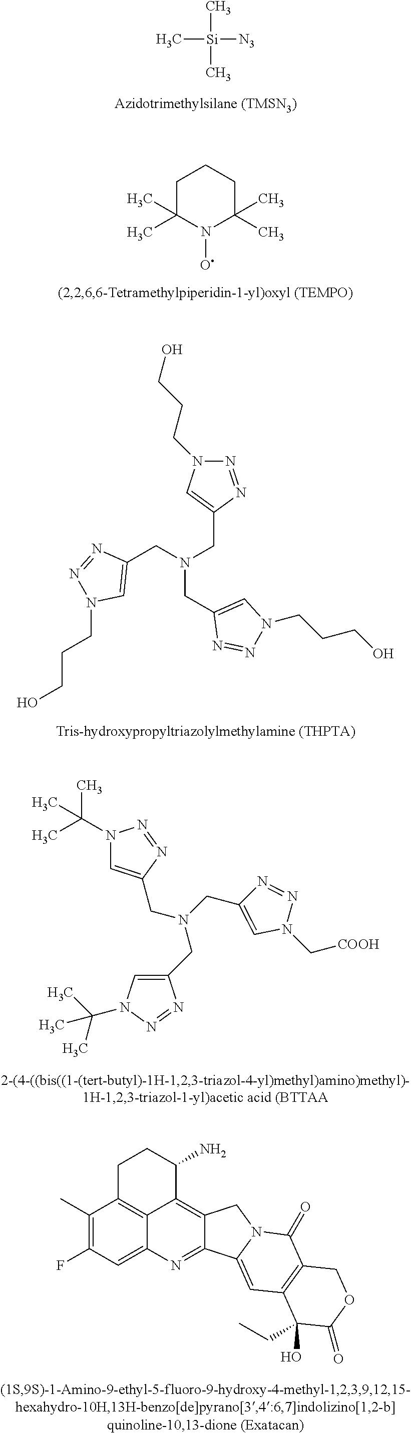

The term “biomolecule” is defined broadly herein to encompass both small and macromolecular molecules found in nature, explicitly including, but not limited to proteins and polypeptides (terms which are used synonymously herein) and polynucleic acids of all types (e.g., DNA, RNA, and combinations thereof). Also included within the term are non-natural modified versions thereof, such as proteins with non-natural residues, tagged and labeled versions of natural biomolecules, etc. Non-limiting examples of “biomolecules” include antibodies, serum proteins, membrane-bound proteins, intracellular proteins and nucleic acids, genomic DNA, mRNA, tRNA, shRNA, etc.

The term “contacting” refers to the act of touching, making contact, or of bringing to immediate or close proximity, including at the molecular level, for example, to bring about a chemical reaction, or a physical change, e.g., in a solution or in a reaction mixture. An “effective amount” refers to an amount of a chemical or reagent effective to facilitate a chemical reaction between two or more reaction components, and/or to bring about a recited effect. Thus, an “effective amount” generally means an amount that provides the desired effect.

The terms “label” and “labeled” are defined broadly herein to encompass any and all molecular markers, labels, or probes of any structure or configuration, now known or developed in the future, that can be detected by any means (now known or developed in the future). The term “label” as used herein is synonymous terms such as “marker” and “probe” and others that are conventionally encountered in the relevant literature. The term “label” includes, without limitation, radioactive labels, fluorescent labels, chromophoric labels, affinity-based labels (such as antibody-type markers, biotin, etc.), and the like. Conventional radioactive isotopes used for detection include, without limitation, ³²p, ¹³C, ²H, and many others. A huge number of fluorescent and chromophoric probes are known in the art and commercially available from numerous worldwide suppliers, including Life Technologies (Carlsbad, California, USA), Enzo Life Sciences (Farmingdale, New York, USA), and Millipore Sigma (also known as Sigma-Aldrich (St. Louis, Missouri, USA).

The term “solvent” refers to any liquid that can dissolve a compound to form a solution, without limitation. Solvents include water and various organic solvents, such as hydrocarbon solvents, for example, alkanes and aryl solvents, as well as halo-alkane solvents. Examples include hexanes, benzene, toluene, xylenes, chloroform, methylene chloride, dichloroethane, and alcoholic solvents such as methanol, ethanol, propanol, isopropanol, and linear or branched (sec or tert) butanol, and the like. Aprotic solvents that can be used in the method include, but are not limited to perfluorohexane, a,a,a-trifluorotoluene, pentane, hexane, cyclohexane, methylcyclohexane, decalin, dioxane, carbon tetrachloride, freon-11, benzene, toluene, triethyl amine, carbon disulfide, diisopropyl ether, diethyl ether, t-butyl methyl ether (MTBE), chloroform, ethyl acetate, 1,2-dimethoxyethane (glyme), 2-methoxyethyl ether (diglyme), tetrahydrofuran (THF), methylene chloride, pyridine, 2-butanone (MEK), acetone, hexamethylphosphoramide, N-methylpyrrolidinone (NMP), nitromethane, dimethylformamide (DMF), acetonitrile, sulfolane, dimethyl sulfoxide (DMSO), propylene carbonate, and the like.

As used herein, the term “click chemistry” or “click reaction” is used to refer generically and broadly to a family of azide-alkyne cyclo-addition reactions, including (by way of example and not limitation) copper(I)-catalyzed azide-alkyne cycloaddition (hereinafter “CuAAC”) and strain-promoted azide-alkyne cycloaddition (hereinafter “SPAAC”), which does not require a copper(I) containing catalyst.

Click reactions are a subset of bioorthogonal reactions that can occur inside living systems without interfering with native biological processes. The term “biorthogonal” implies that the reaction does not react with or disrupt endogenous biomolecules present in biological systems.

The CuAAC reaction has been widely reported in scientific literature. See, for example, Presolski, Hong, and Finn (2011) “Copper-Catalyzed Azide-Alkyne Click Chemistry for Bioconjugation,” Curr Protoc Chem Biol. 3(4):153-162. The CuAAC reaction proceeds generally by the following reaction scheme:

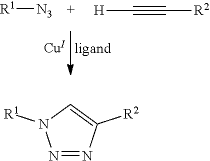

The basic CuAAC reaction requires only copper ions in the +1 oxidation state. These may be supplied by a discrete Cu(I) complex, by metallic copper, or copper-impregnated materials. See, for example, Rostovtsev V V, Green L G, Fokin V V, Sharpless K B. (2002) “A Stepwise Huisgen Cycloaddition Process: Copper(I)-Catalyzed Regioselective Ligation of Azides and Terminal Alkynes,” Angew Chem, Int Ed. 41:2596-2599. See also Lipshutz B H, Frieman B A, Tomaso A E., J (2006) “Copper-in-Charcoal (Cu/C): Heterogeneous, Copper-Catalyzed Asymmetric Hydrosilylations,” Angew Chem, Int Ed. 45:1259-1264. The reaction is also widely practiced using a mixture of a Cu(II) salt and a reducing agent, sodium ascorbate being the most popular (see, e.g., Rostovtsev et al. 2002, supra). Optionally, accelerating ligands may also be added to the reaction. These accelerating ligands act as chelating agents to maintain a readily available concentration of Cu(I) in solution. (Copper ions are quite facile and can undergo redox and disproportionation reactions that rapidly decrease the concentration of Cu(I) in the reaction solution.)

The CuAAC reaction has a host of benefits in the context of conjugating biomolecules. It yields a non-toxic triazole from biological building blocks that have been modified with non-perturbing azides and unactivated alkynes. The CuAAC reaction is reliable and tolerates a wide range of reaction conditions. It is pH-independent and can be carried out in water at ambient, room temperature. It can be utilized in reactions taking place entirely in solution and can also be utilized for solid-phase immobilization reactions. In the biomolecular realm in particular, azido groups and acetylenic groups are quite rare in natural biomolecules. Hence, the reaction is highly bio-orthogonal and specific.

The Cu(I)-free [2+3] cycloaddition, strain-promoted click strategy (SPAAC) relies on the use of strained dibenzylcyclooctynes (“DBCO's”). See, for example, Agard N J, Prescher, J A, and Bertozzi, CR (2004) “A strain-promoted [3+2] azide-alkyne cycloaddition for covalent modification of biomolecules in living systems,” J. Am. Chem. Soc. 126(46):15046-7. The strained conformation of DBCO's decreases the activation energy for the cycloaddition click reaction, enabling it to be carried out without the need for a catalyst. The reactions take place at low temperatures (ambient) with an efficiency greater than that of the Cu(I)-catalyzed ligation.

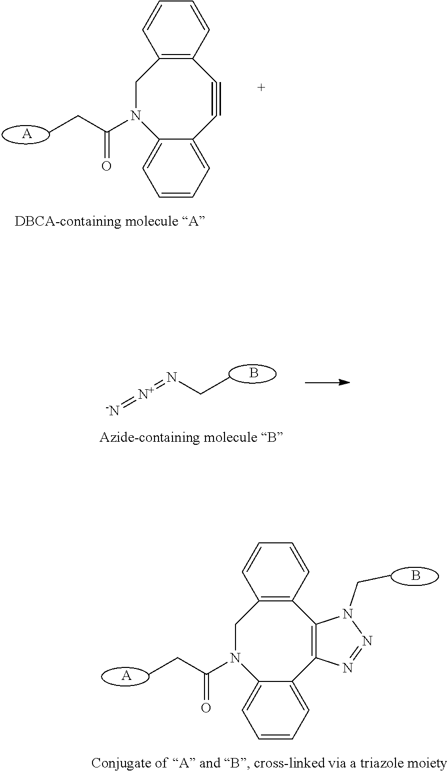

The Cu(I)-free ligation reaction scheme is shown schematically above. Diarylcyclooctyne-activated biomolecule A reacts with azide-activated biomolecule B without Cu(I) in aqueous conditions to form a stable triazole. Diarylcyclooctynes are thermally stable compounds with very narrow and specific reactivity toward azides. The ligation reaction is very fast and results in almost quantitative yield of stable triazoles.

The flexibility and utility of copper-free click chemistry in the investigation of the interactions of biomolecules is manifest. For example, a novel class of difluorinated cyclooctyne (DIFO) reagents were employed in copper-free click chemistry for the site-selective labeling of biomolecules in vitro and in vivo. See Codelli J A, Baskin J M, Agard N J, Bertozzi C R. (2008) “Second-Generation Difluorinated Cyclooctynes for Copper-Free Click Chemistry,” J. Am. Chem. Soc. 130(34):11486-11493.

Catalyst-free click reactions are useful for preparing radiometal-based pharmaceuticals. Radiotracer [⁶⁴Cu]DOTA-ADIBON₃-Ala-PEG₂₈-A20FMDV2, used for positron emission tomography imaging of integrin ανβ6-expressing tumors, has been synthesized via copper-free click chemistry. Satpati D, Bauer N, Hausner S H, Sutcliffe J L (2014) “Synthesis of [⁶⁴Cu]DOTA-ADIBON₃-Ala-PEG₂₈-A20FMDV2 via copper-free click chemistry for PET imaging of integrin ανβ6,” J Radioanal Nucl Chem. 302(2):765-771.

Iodine radioisotope labeling of cyclooctyne-containing molecules by copper-free click reaction has been reported. Radioiodination using the tin precursor was carried out at room temperature to obtain ¹²⁵I-labeled azide. Dibenzocyclooctyne (DBCO)-containing cRGD peptide and gold nanoparticle were labeled by employing ¹²⁵I-labeled azide to afford triazoles in good radiochemical yields (67-95%). This method is useful for both in vitro and in vivo labeling of DBCO group-containing molecules with iodine radioisotopes. See Jeon J, Kang J A, Shim H E, Nam Y R, Yoon S, Kim H R, Lee D E, Park S H (2015) “Efficient method for iodine radioisotope labeling of cyclooctyne-containing molecules using strain-promoted copper-free click reaction,” Bioorganic & Medicinal Chemistry 23(13):3303-3308.

A protein, site-specific labeling techniques employing the SPAAC reaction between dibenzocyclooctyne-fluor 545 (DBCO-fluor 545) and an azide-bearing unnatural amino acid is described in Zhang G, Zheng S, Liu H, Chen P R. (2015) “Illuminating biological processes through site-specific protein labeling,” Chem. Soc. Rev. 44 (11): 3405-3417.

These types of click chemistry reactions, and many more, are made much easier using the presently disclosed method because the method allows for direct and controllable azidylation of a biomolecule of interest.

A wide range of reagents for practicing click chemistry are available commercially from numerous international suppliers, including Millipore-Sigma, Inc. (Madison, Wisconsin, USA, a wholly owned subsidiary of Merck KGaA, Darmstadt, Germany), Interchim Inc. (San Pedro, California, USA), Interchim SA (Montluçon, France), and Cheshire Sciences Ltd., Chester, England. These reagents include a host of labeled alkynes and azides that permit a huge array of discovery-type and confirmatory-type reactions. These commercially available reagents include fluorescently labeled alkynes and azides, biotin-tagged alkynes and azide, and the like.

### Biomolecule Azidylation

Disclosed herein is a simpler method for rapidly attaching azide (N₃) groups to biomolecules (e.g., proteins) in solution. In a previously developed azidylation method, an oxidative reagent is required (see WO2023/122242 A1). The present disclosure has discovered that direct protein azidylation requires only copper(I), azide, and protein, and the reaction appears to specifically modify histidine residues.

The method disclosed herein has potential for better controllable protein modification as compared to the original reaction, to facilitate click chemistry directly on proteins. This is advantageous because getting an alkyne or an azide (each ½ of the click reaction copper-catalyzed azide-alkyne cycloaddition) onto a protein is the hard part of enabling click-based conjugation. Synthesizing alkyne-containing compounds is generally preferred to azide-containing compounds due to the hazardous nature of azides. But the method disclosed herein is a safe reaction. The amounts of azide used are minimal, and the chemistry presents no safety issues for a general life scientist trained in basic lab safety. Additionally, the method allows for controllable protein modification which is more desirable for the antibody-drug conjugate use. If one can get an azide onto a protein and available for downstream conjugation, virtually any alkyne can be covalently bound, and the formed linkage between the azide and alkyne is effectively irreversible. Thus, there is promise here for activating protein for conjugation of all sorts, with many biological and chemical applications.

In the present disclosure, the azidylation method was tested using trastuzumab as an exemplary biomolecule. Various reaction conditions were investigated, including the form and source of Cu, the source of azide, buffers, solvents, concentration of reagents, reaction time, the order of adding reagents, the method of reducing protein degradation, and subsequent click reactions.

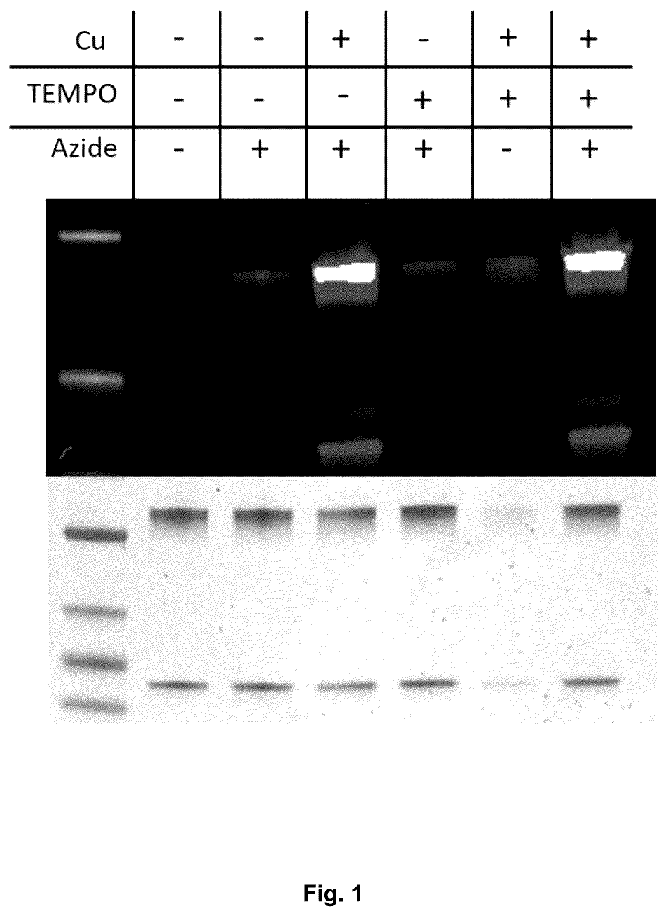

In an initial test, the molecule TEMPO (a stable free radical) was used as a radical scavenger/initiator, but is shown to be unnecessary. As shown in FIG. 1, Cu(I) and azide are sufficient for azide addition. The reaction without TEMPO (lane 3) resulted in similar level of azide addition compared to the reaction with TEMPO (lane 6). Azide addition did not occur in tests lacking one or both of Cu(I) and azide (lanes 1, 2, 4, and 5). TEMPO may mitigate a bit of degradation, but is not required. In this test, NaN₃ was used as the source of azide, and Cu(I) Cl was used as the source of Cu(I). It is noted that the concentration of NaN₃ used here is 10 mM, which is significantly lower than previous methods typically using 100 mM and above.

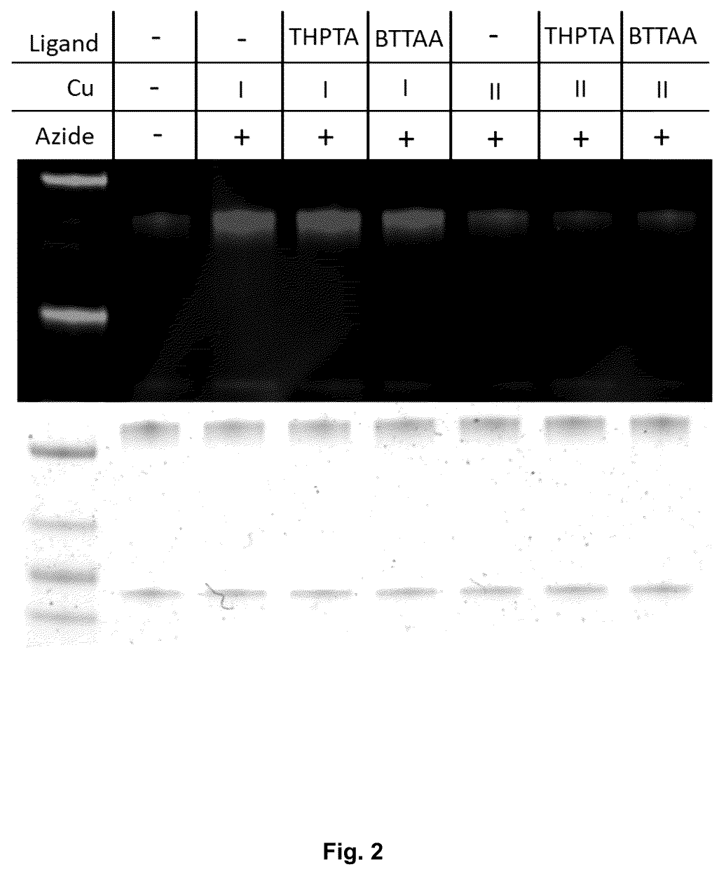

Subsequently, Cu(II) and ligand-bound Cu(I) were tested. As shown in FIG. 2, Cu(I) is needed for azide addition and clicking above background levels. The reactions with Cu(I) (lanes 2-4) resulted in significantly higher levels of azide addition compared to the reactions with Cu(II) (lanes 5-7). Cu(II) may result in a little bit of extra addition in lane 5, but it's not as marked as with Cu(I). The Cu(I) with or without ligand-bound yielded similar results. Here, the ligands THPTA and BTTAA were tested (lane 2: no ligand; lane 3: THPTA; lane 4: BTTAA).

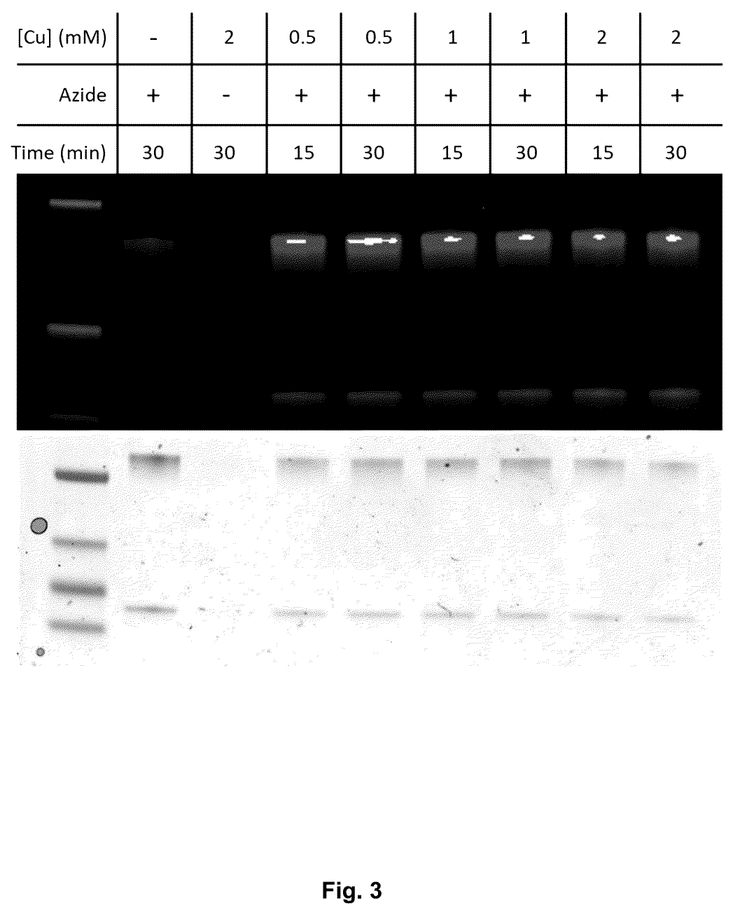

Reactions with Cu(I) at concentrations of 0.5, 1, and 2 mM and a reaction time of 15 and 30 min were tested, and the results are shown in FIG. 3. As shown in the figure, 0.5 mM and 15 min are sufficient for the reaction (lane 3), indicating that the reaction can occur quickly. The level of azide addition under this condition (lane 3) shows no apparent difference from reactions with higher concentrations of Cu and/or a longer reaction time (lanes 4-8). The results also show that Cu alone (lane 2, without azide) caused protein degradation.

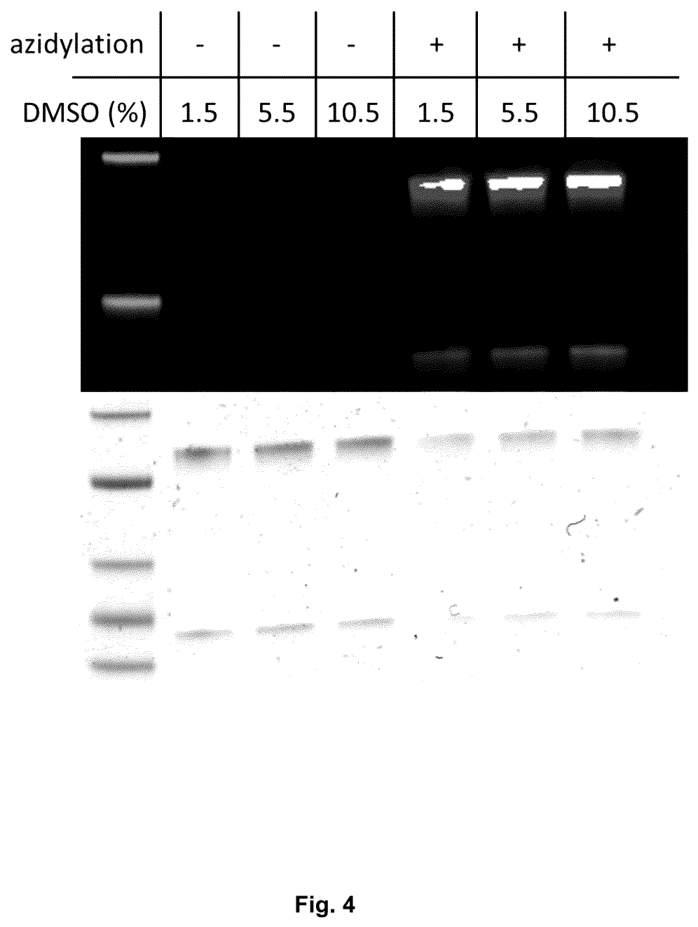

In the above tests, acetonitrile was used as the solvent. Further tests show that the reaction is compatible with other organic solvents, such as DMSO. FIG. 4 shows successful azide addition with 1.5%, 5.5%, and 10.5% DMSO (lanes 4-6). This is important as Cu(I) compounds are not that soluble in aqueous solvents. Additionally, DMSO may prevent some degradation of protein.

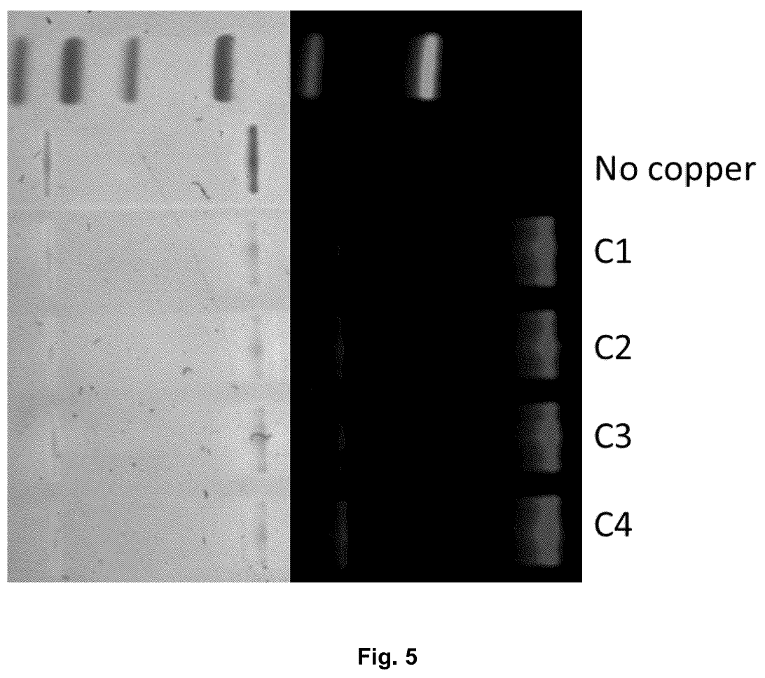

Four compounds of Cu(I) were further tested, including tetrakis(acetonitrile)copper(I) hexafluorophosphate (C1), tetrakis(acetonitrile)copper(I) trifluoromethanesulfonate (C2), copper(I) iodide (C3), and copper(I) chloride (C4). As shown in FIG. 5, all the four forms of Cu(I) are suitable for use in the azidylation reaction.

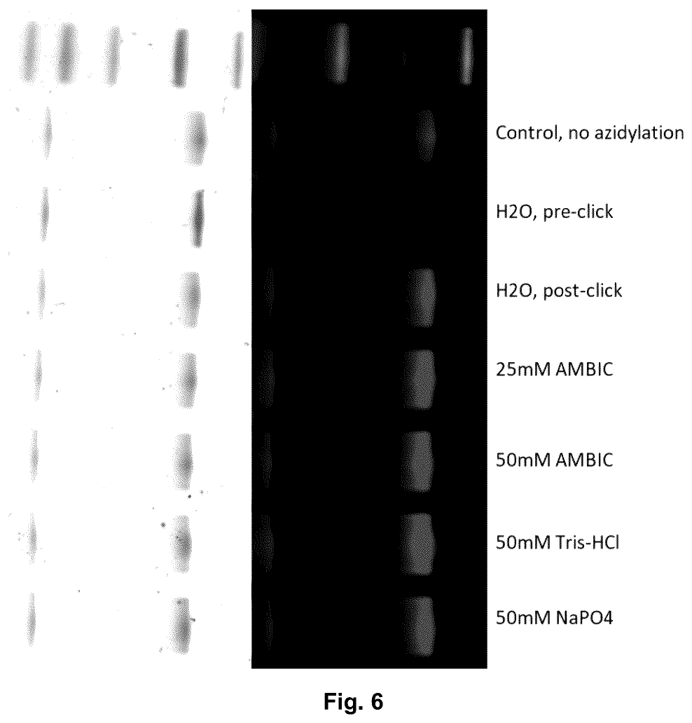

Different buffer systems of the reaction were also tested, including water, 25 or 50 mM ammonium bicarbonate (AMBIC), 50 mM Tris-HCl, and 50 mM sodium phosphate (NaPO₄). As shown in FIG. 6, all the tested buffers work for the reaction, with Tris buffer yielding the best result. The test also showed that clicking itself is causing protein degradation and the observed background. See the pre-and post-click lanes (lanes 2 and 3). Using minimal reagent conditions for clicking could help to mitigate the degradation.

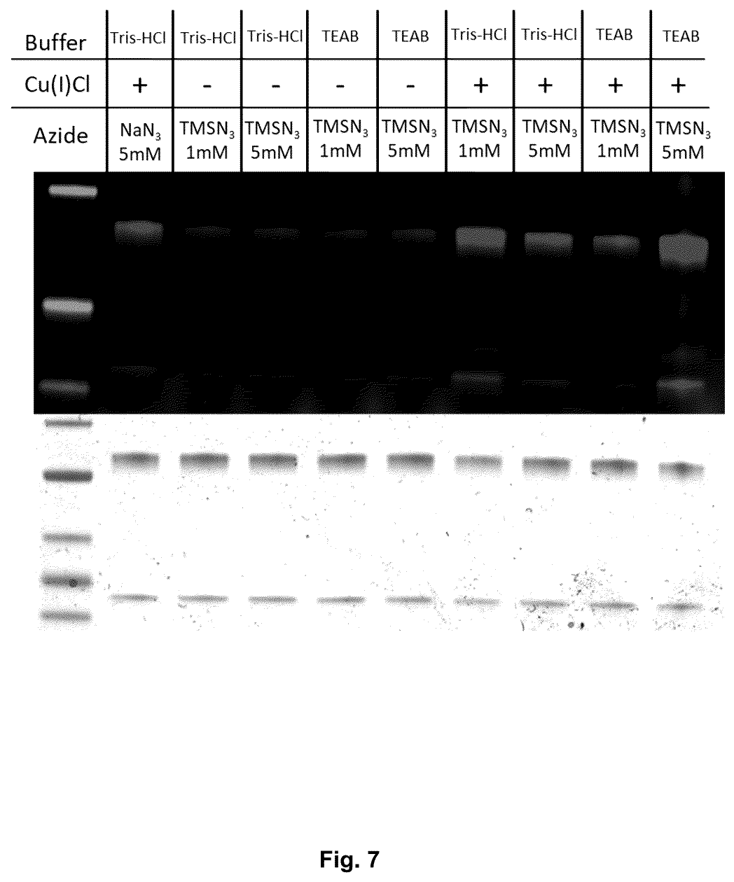

Other than NaN₃, azidotrimethylsilane (TMSN₃) was tested as an azide donor, with Tris-HCl or triethyl ammonium bicarbonate (TEAB) as background buffer. As shown in FIG. 7, TMSN₃ appears to be a better azide donor (comparing lanes 6-9 with TMSN₃ to lane 1 with NaN₃). The results show a clear Cu-dependent reaction, as azide addition did not occur without Cu(I) (see lanes 2-5). Some degradation of the protein was observed and seemed to be correlated with the modification.

TMSN₃ tends to work better over shorter reaction times (e.g., a 1-hour reaction), while sodium azide (NaN₃) tends to work better over longer reactions times (e.g., a 24-hour reaction).

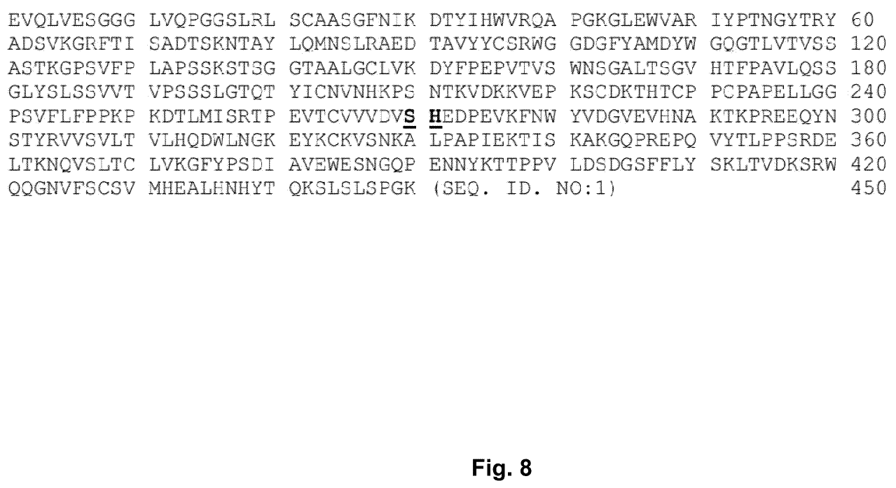

The direct azide addition and clicking on of a derivatized drug alkyne was confirmed by mass spectrometry. See FIG. 8 and SEQ ID. NO: 1. The reaction is shown below:

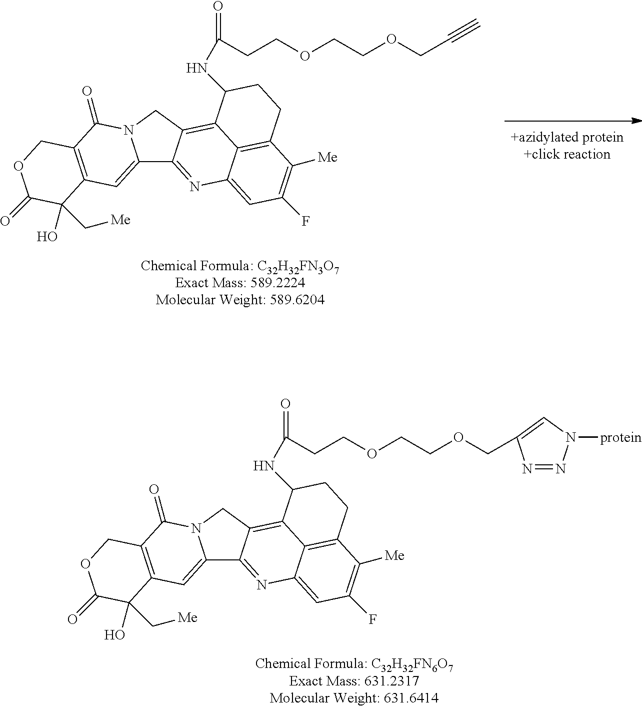

As shown by the bolded residues in FIG. 8, the modification was attached at residues 271 and 272 of the protein. (For the starting material, Exatacan, see U.S. Pat. No. 5,834,476, issued Nov. 10, 1998, to Terasawa et al.)

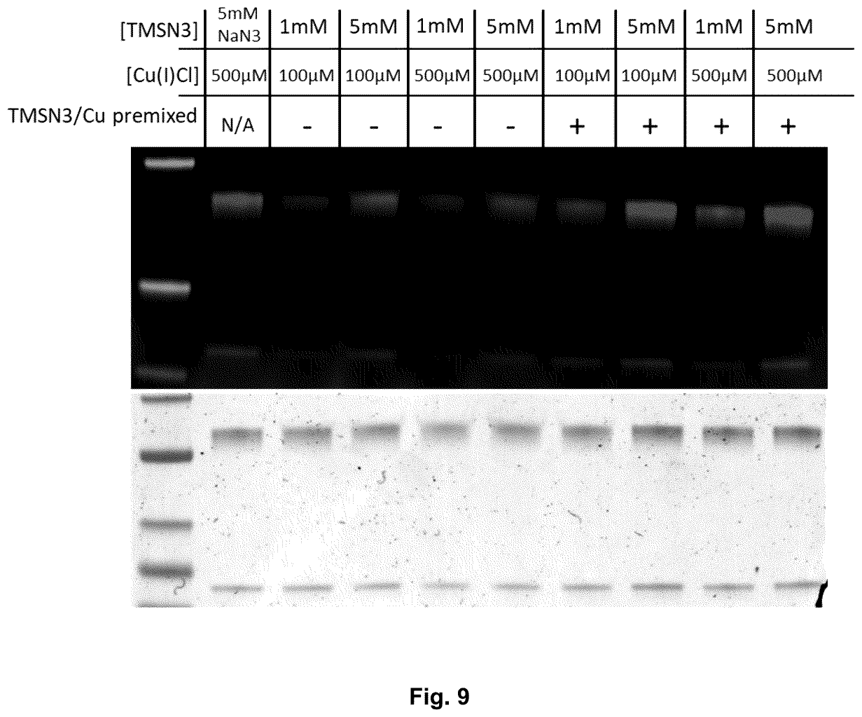

The order of adding matters in the reaction was tested by premixing Cu(I)Cl and TMSN₃ before adding to protein versus adding Cu(I)Cl first for 1 min, then adding TMSN₃. The results were compared at different TMSN₃ concentrations (1 mM vs. 5 mM) and also compared to the reaction with NaN₃ as the azide donor. As shown in FIG. 9, premixing Cu(I) with TMSN₃ appears to yield more modification. TMSN₃ appears to be more important than Cu. To that point, 100 μM Cu premixed with 1 or 5 mM TMSN₃ appeared to give good modification, more than NaN₃.

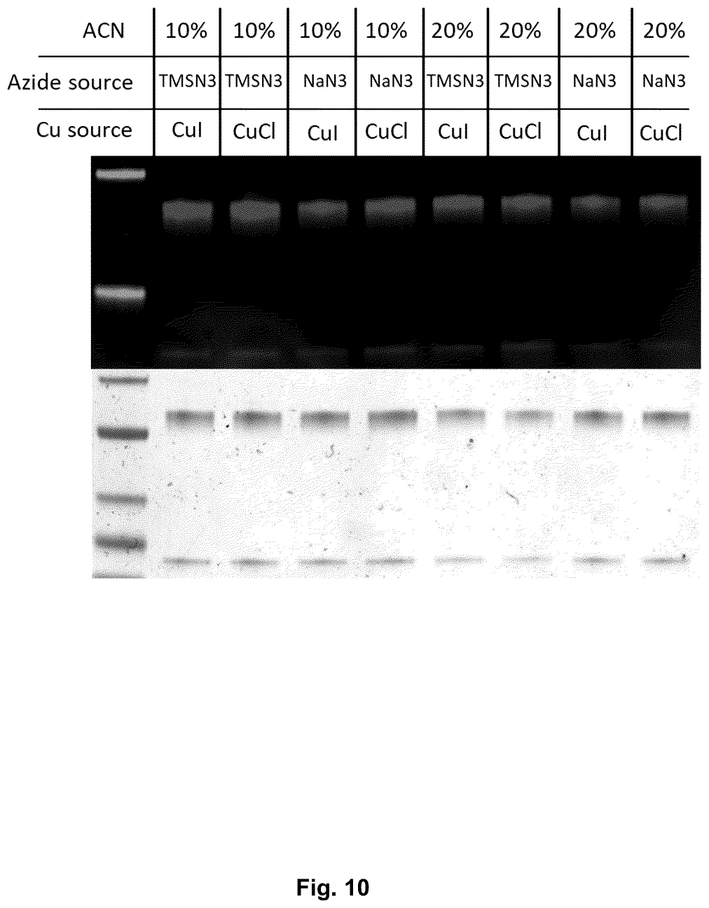

We further tested premixed azide (TMSN₃ or NaN₃) with CuI or CuCl in 10% or 20% acetonitrile. As shown in FIG. 10, higher concentration of organic solvent does not lead to more modification; rather, higher organic inhibits the reaction (see lanes 1-4 with 10% acetonitrile vs. lanes 5-8 with 20% acetonitrile). CuI was shown to work as well as CuCl. As CuI is more soluble in acetonitrile, subsequent tests were conducted using CuI. The results also confirmed that TMSN₃ appears to be a better azide donor than NaN₃.

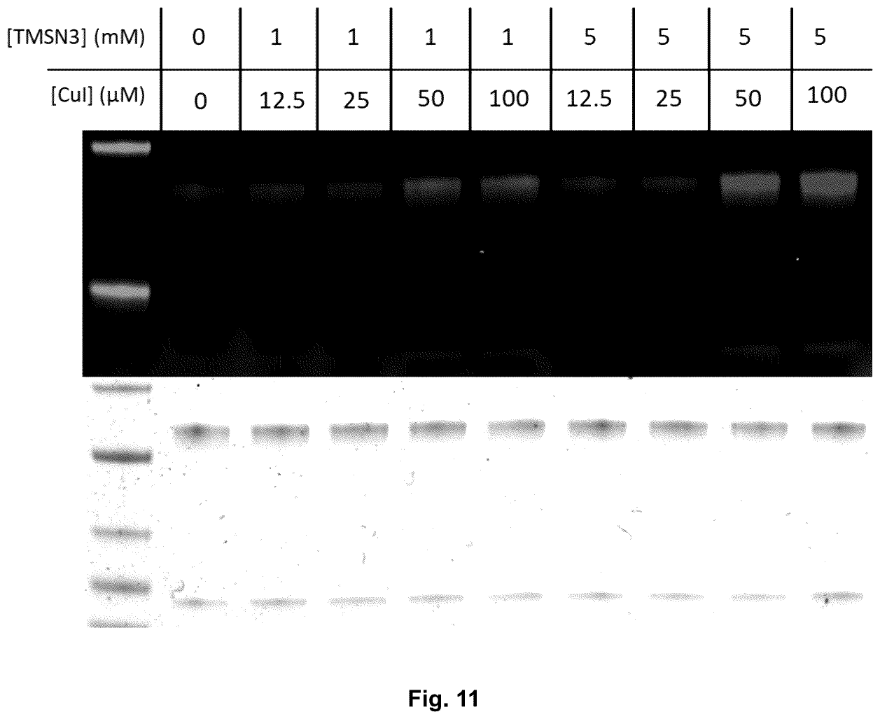

The tests have repeatedly shown that adding more Cu is detrimental, as it clearly degrades protein, and in general, excess Cu can bind to and disrupt protein structure. Minimizing Cu concentration could help to reduce protein degradation. Our test showed that Cu(I) can be minimized to about 1:40 protein:Cu molar ratio. As shown in FIG. 11, there appears to be a threshold effect at about 50 μM Cu. Azide addition did not occur with Cu concentrations lower than 50 μM (see lanes 2, 3. 6, and 7 with 12.5 or 25 μM Cu). Also, it is clear from the results that higher concentrations of TMSN₃ resulted in more modification.

However, lowering Cu concentration also lowers the yield. There is a balance between protein modification with protein degradation. As modification goes up, so as degradation. Therefore, we sought to gently increase the amount of modification without adding excesses of Cu(I) and shredding the protein. Two ways for gently augmenting the reaction were explored:

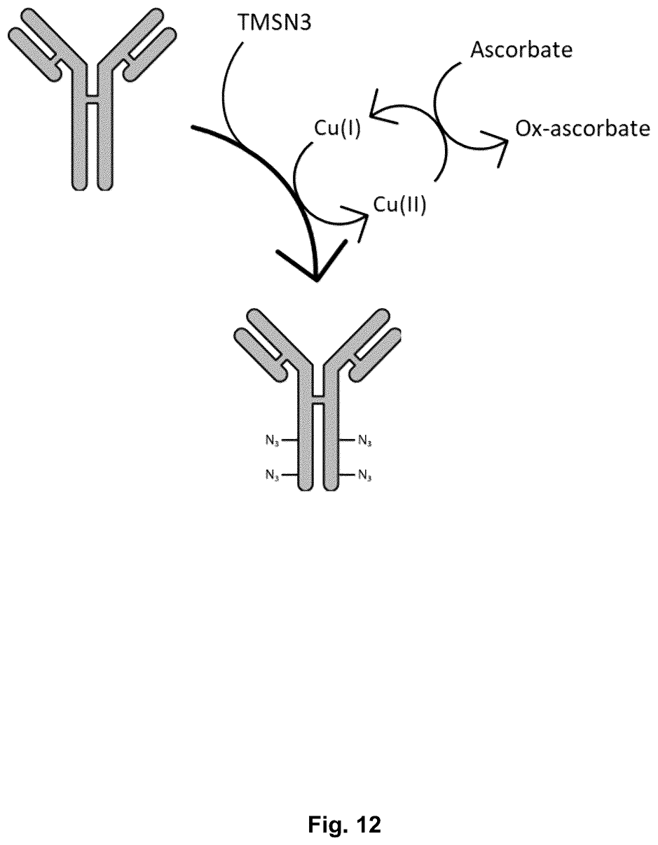

First, adding a reductant, such as ascorbate, was tested to verify if it could regenerate Cu(I) from Cu(II) in situ (the reaction is shown below and in FIG. 12). This would enable adding much less Cu, as Cu(II) is water soluble, whereas basically no Cu(I) compounds are. Additionally, Cu(II) has no solubility/background oxidation issues. This way, increasing the ascorbate dose would increase the effective Cu(I) dose given, over the time of the reaction. Ascorbate should not affect the protein itself, and it is an antioxidant. Our results have shown that there is no difference between adding Cu(I) and Cu(II), if Cu(I) is generated in situ from Cu(II).

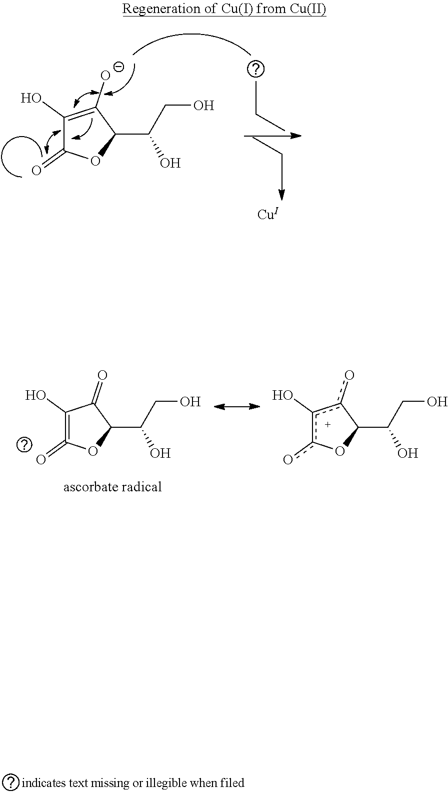

Second, adding a Cu ligand was tested for coordination and to prevent the Cu causing protein degradation. Cu is less likely to bind protein when it is bound to a ligand. The ligand is typically an antioxidant and absorbs some amount of oxidation. The ligand also helps with Cu(I) solubility. With the presence of the ligand, when Cu(I) is generated from the reduction of Cu(II), it does not crash out of solution and is readily available to react. As with ascorbate alone, Cu ligand is innocuous to protein structure.

Our results have shown that with the reductant and Cu ligand, azide addition can be conducted at high levels, with significantly less protein degradation.

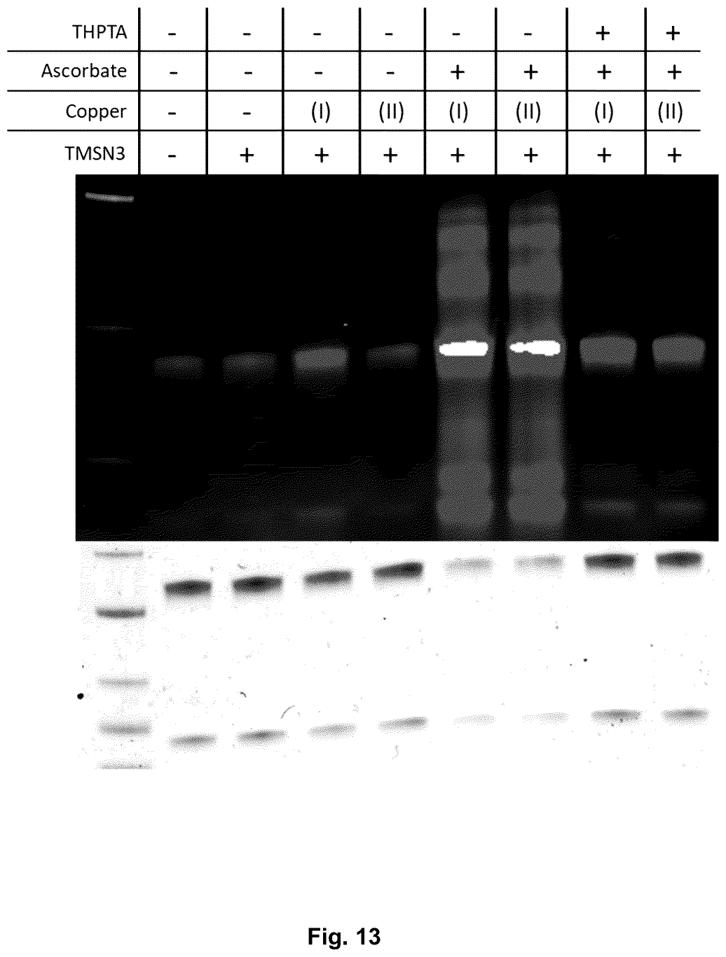

In one test, sodium ascorbate was added to generate Cu(I) from Cu(II) in situ, and a Cu-coordinating ligand THPTA was added to Cu prior to adding to reaction. The reaction was run for 30 min. The results were compared to conditions without ascorbate and/or without THPTA. The results as shown in FIG. 13 were consistent with previous finding that the reaction is Cu(I) dependent, and does not work with Cu(II). As shown in lane 4, modification did not occur with Cu(II) but without ascorbate. Both Cu(I) and TMSN₃ are necessary for azidylation above background levels. Adding ascorbate to generate Cu(I) from Cu(II) in situ amplifies the reaction significantly, but also causes protein degradation (see lanes 5-6 without adding THPTA). In contrast, adding the Cu-coordinating ligand THPTA on top of that prevents this degradation, while increasing the modification (lanes 7-8).

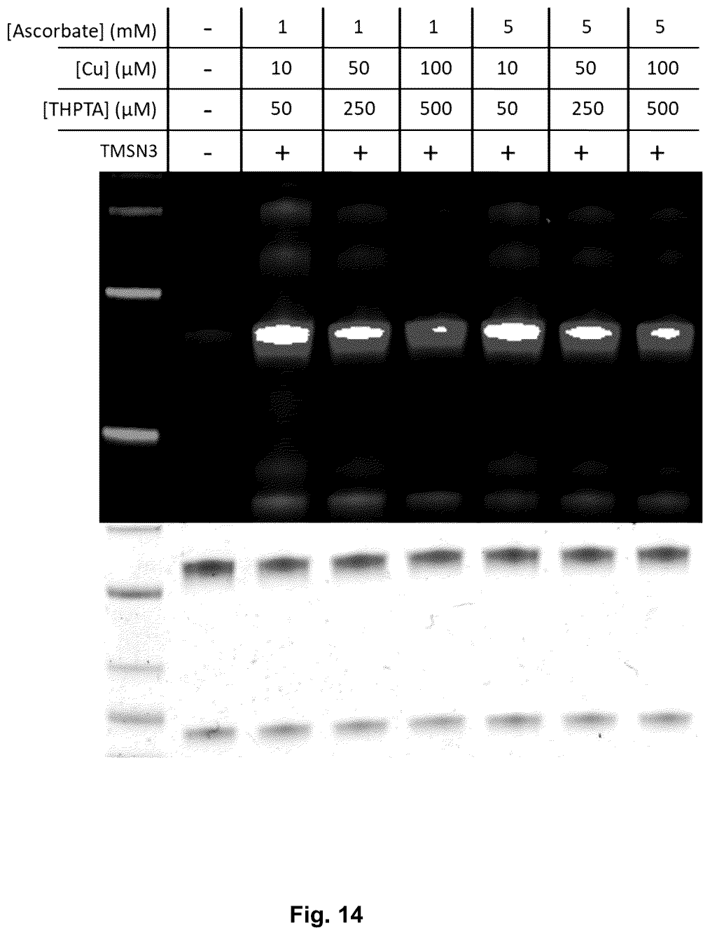

Different concentrations of ascorbate (1 and 5 mM), Cu(II) (10, 50, and 100 μM), and THPTA (50, 250, and 500 μM) added to the reaction were further investigated, along with prolonged reaction time (60 min). The Cu:THPTA ratio was kept constant at 1:5. As shown in FIG. 14, the azide addition reaction works better with less added ligand-bound Cu and more reaction time. Down to as little as 10 μM Cu (about 7:1 Cu:Protein ratio) with ascorbate regeneration leads to significant modification (lanes 2 and 5). Protein degradation is much less than without THPTA added. The results also show that more Cu and/or THPTA appear to inhibit the reaction (see lanes 2 and 5 with 10 M Cu and 50 μM THPTA compared to higher concentrations of Cu and THPTA in lanes 3-4 and 6-7). There appears to be no apparent difference between 1 mM and 5 mM ascorbate, and thus the ascorbate concentration could probably be lessened further.

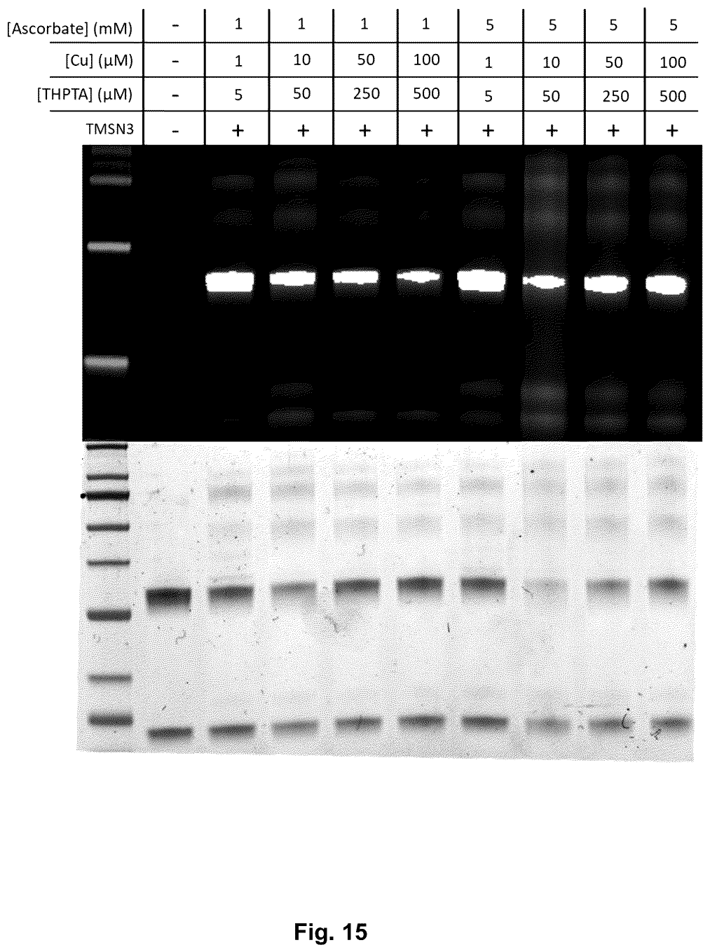

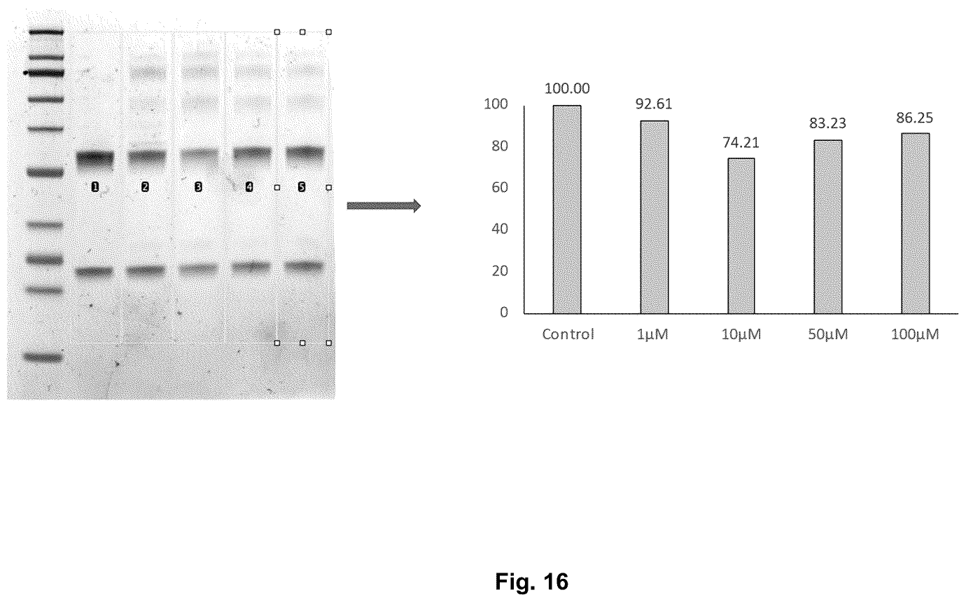

In a subsequent test, the Cu(II) and THPTA concentrations were further lowered to 1 μM and 5 μM, respectively, and the reaction time was further prolonged to 17 hours. The Cu:THPTA ratio was still kept constant at 1:5. As shown in FIG. 15, when left longer, less Cu and THPTA concentrations appear to better balance the modification and degradation (see lanes 2 and 6 with 1 μM Cu and 5 μM THPTA compared to higher concentrations of Cu and THPTA in lanes 3-5 and 7-9). Less ascorbate appears to better balance degradation with modification (see lanes 2-5 with 1 mM ascorbate compared to lanes 6-9 with 5 mM ascorbate). FIG. 16 shows relative intensities of bands in lanes 1-5 of FIG. 15. About 93% of signal remains with 1 μM Cu, much higher than the results with 10, 50, or 100 μM Cu. Induced crosslinking (presumably due to oxidation) was seen with this longer timepoint reaction with 17 hours. It is hard to untangle degradation and oligomerization here. In some lanes, degradation may be minimal, and loss of the monomeric band may be due to crosslinking and oligomerization, though in some lanes it is clearly both. Overall, the results suggest that if decreasing the reactants, the reaction can run overnight without totally shredding protein.

### Click Reaction

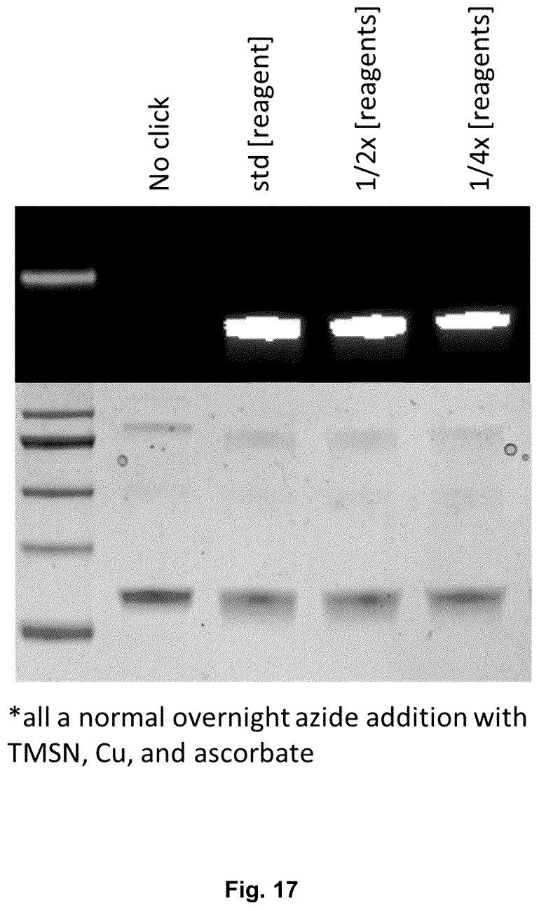

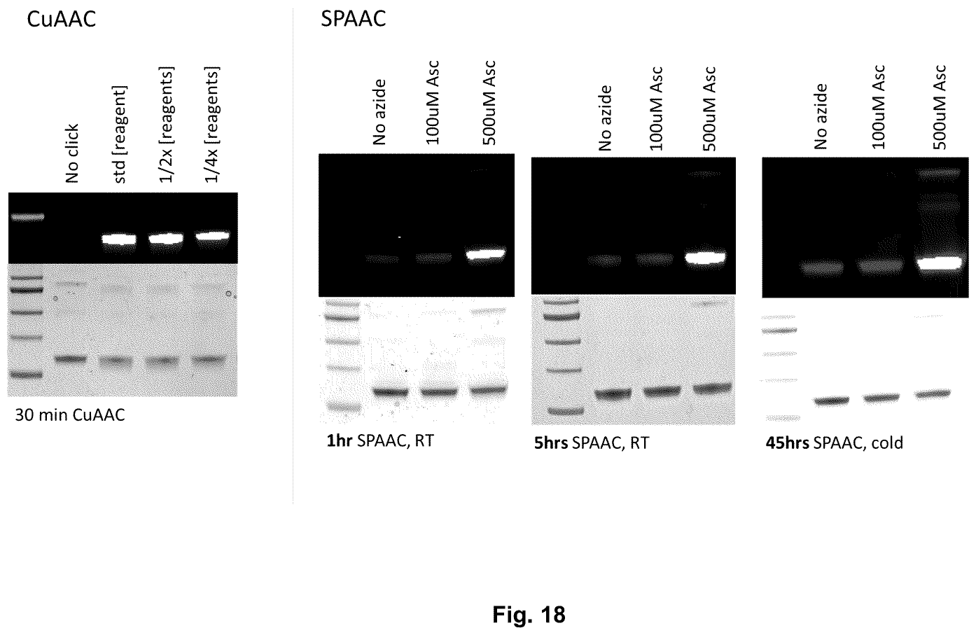

The azidylated biomolecule generated from the above-described method can subsequently react with a reagent comprising an alkyne via CuAAC or SPAAC reaction. It is found that the CuAAC reaction can cause band smearing, as shown in FIG. 17. We further tested the SPAAC reaction with the azidylated protein, and the reaction was run for 1, 5, or 45 hours. As shown in FIG. 18, the SPAAC click reaction reduces smearing compared to CuAAC clicking. The results also show that the SPAAC reaction can be left for a prolonged time (e.g., 45 hours) without negative effects.

### Example

As shown in the following example, histidine residues are the major site of azidylation. The question was asked: Will a protein with a 6× C-terminal His-tag get modified more heavily than a protein without one? This is relevant because His-tags are used extensively for protein purification via affinity chromatography. A host of proteins can be purchased commercially that are pre-labelled with a His-tag. The ability to further modify a His-tag using click-chemistry would be extremely useful.

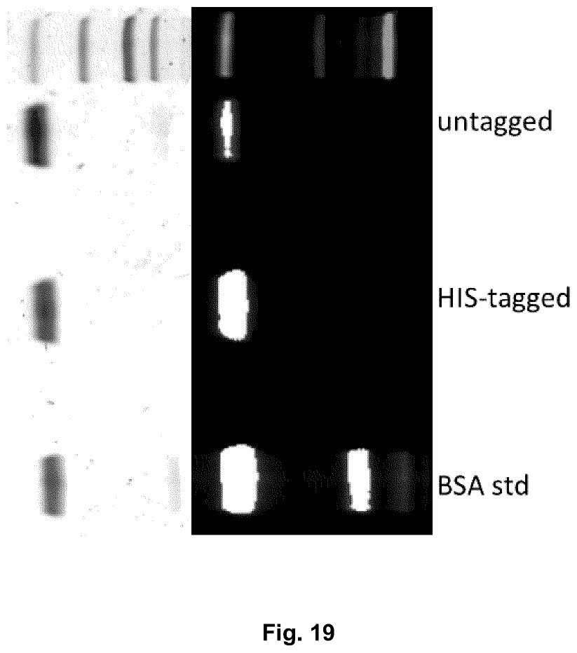

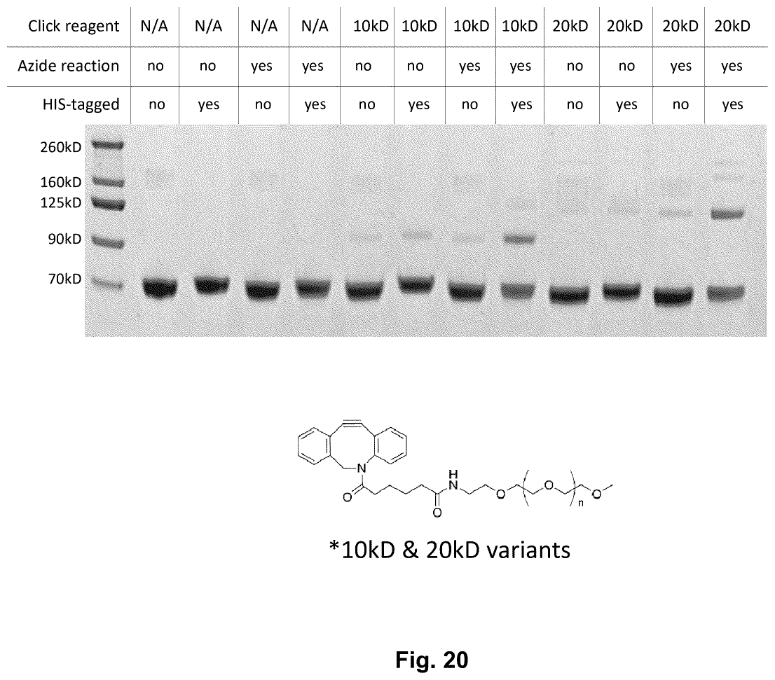

To evaluate the question, unmodified and 6× His-tagged human serum albumin (HSA) were purchased from Abcam, Waltham, Massachusetts: Recombinant Human Serum Albumin protein (His tag), catalog no. ab217817, and Native Human Serum Albumin protein, catalog no. ab205808. The sequence of these two proteins is identical with the exception of a 6× His-tab on the C-terminus. The results are shown in FIGS. 19 and 20.

Referring now to FIG. 19, the figure clearly shows that His-tagged HSA was modified more heavily than untagged protein. The reaction conditions for the azidylation/click reaction were as follows:
˜ 3-4 μM HSA, tagged or untagged
50 mM triethyl ammonium bicarbonate (TEAB) as background buffer, ˜pH 6.8- 7.0
5 mM NaN₃
1 μM Cu(II)/50 μM THPTA (tris-hydroxypropyltriazolylmethylamine—a Cu ligand)
500 μM sodium ascorbate.
Reaction run time: ˜18-20 hours, in the cold and dark

In FIG. 19, the far-right lane is bovine serum albumin (BSA) pre-conjugated with azide. This was used as a positive control. As is clearly seen in the figure, the His-tagged protein picks up more fluorescence than the untagged protein.

The above experiment was then run again under various conditions using DBCO-PEG click reagents. The results are shown in FIG. 20. The reaction conditions for the azidylation/click reaction were as follows:
˜3-4 μM HSA, tagged or untagged.
50 mM Triethyl ammonium bicarbonate (TEAB) as background buffer, ˜pH 6.8-7.0
+/−5 mM NaN₃
+/−1 μM Cu(II)/50 μM THPTA
+/−500 M sodium ascorbate.
Reaction run time: ˜18-20 hours, in the cold and dark.
Clicked the DBCO-PEG reagents shown in FIG. 20 for ˜3 hours.

The results here mirror those shown in FIG. 19. Labelling can be quantified: For the His-tagged protein, ˜33% was converted to the clicked form, using either the 10- or 20-kD click reagents. The major form of the product is a +2 adduct, with a shift of 20 and 40 kD respectively.

Thus, with both clicked on fluorescence (FIG. 19) and DBCO-PEG reagents (FIG. 20), a His-tag on the protein increases labeling.

What is claimed is:

1. A method of modifying a biomolecule, the method comprising:
attaching an azide moiety to a biomolecule by contacting the biomolecule in a solution with an azide and a copper-containing reagent, and in the absence of added oxidizing agent, for a time wherein at least one azide moiety is covalently bonded to the biomolecule to yield an azidylated biomolecule.

2. The method of claim 1, wherein the copper-containing reagent comprises copper(I).

3. The method of claim 1, wherein the copper-containing reagent comprises copper(II), and the solution comprises a reductant to generate copper(I) ions from copper(II).

4. The method of claim 1, wherein the solution comprises a ligand to coordinate copper ions.

5. The method of claim 1, wherein the biomolecule is a protein.

6. The method of claim 5, wherein the protein is selected from the group consisting of an intracellular protein, a membrane-bound protein, a circulating protein, and an antibody.

7. The method of claim 1, wherein the biomolecule is a nucleic acid polymer.

8. The method of claim 7, wherein the nucleic acid polymer is a DNA polymer.

9. The method of claim 7, wherein the nucleic acid polymer is a RNA polymer.

10. The method of claim 1, comprising contacting the biomolecule with the azide for 1 second to 48 hours.

11. The method of claim 1, further comprising reacting the azidylated biomolecule with a reagent comprising an alkyne.

12. The method of claim 11, wherein the alkyne is a terminal alkyne.

13. The method of claim 11, wherein the alkyne is an internal alkyne.

14. The method of claim 11, wherein the reagent comprising an alkyne is a cyclic alkyne.

15. The method of claim 11, wherein the reaction with the alkyne is a copper-catalyzed azide-alkyne cycloaddition (“CuAAC”) reaction.

16. The method of claim 11, wherein the reaction with the alkyne is a strain-promoted alkyne-azide cycloaddition (“SPAAC”) reaction.

17. A method of modifying a biomolecule, the method comprising:
attaching an azide moiety to a biomolecule by contacting the biomolecule in a solution with an azide and a copper-containing reagent, for a time wherein at least one azide moiety is covalently bonded to the biomolecule to yield an azidylated biomolecule; and
reacting the azidylated biomolecule with a reagent comprising an alkyne.

18. The method of claim 17, wherein the alkyne is a terminal alkyne.

19. The method of claim 17, wherein the alkyne is an internal alkyne.

20. The method of claim 17, wherein the reagent comprising an alkyne is a cyclic alkyne.

21. The method of claim 17, wherein the reaction with the alkyne is a copper-catalyzed azide-alkyne cycloaddition (“CuAAC”) reaction.

22. The method of claim 17. wherein the reaction with the alkyne is a strain-promoted alkyne-azide cycloaddition (“SPAAC”) reaction.
